(19) United States
(12) Patent Application Publication
(10) Pub. No.: US 2026/0098284 A1
(43) Pub. Date: Apr. 9, 2026

(54) CELL-FREE PPi-DRIVEN ATP REGENERATION PLATFORM

(71) Applicant: Stepan Tymoshenko, Sacramento, CA (US)

(72) Inventor: Stepan Tymoshenko, Sacramento, CA (US)

(21) Appl. No.: 19/349,943

(22) Filed: Oct. 3, 2025

Related U.S. Application Data
(60) Provisional application No. 63/702,765, filed on Oct. 3, 2024.

(51) Int. Cl. C12P 9/00; C12M 1/34; C12N 9/00; C12N 9/12; C12N 9/88

(52) U.S. Cl. CPC C12P 9/00; C12M 41/26; C12M 41/32; C12N 9/1229; C12N 9/1294; C12N 9/88; C12N 9/93; C12Y 207/04003; C12Y 207/09001; C12Y 401/01038; C12Y 402/01001; C12Y 604/01001

(57) ABSTRACT

Bio-production processes that rely on biological activity can be very slow and inefficient, yet, the output of these processes may have significant value. Using biological enzymes outside their natural cellular environments offers a significant and largely untapped opportunity to enhance bio-production processes. By decoupling enzymes from their native contexts, one can modify both their sequences and structures in ways that are favorable for industrial applications. This system and method mixes substrate molecules, PPi, and enzymes that utilize PPi (PPi-dependent enzymes) in a reaction chamber and ends with stable-form products, including phosphorylated molecules.

### CROSS-REFERENCE TO RELATED APPLICATION

This application claims the benefit of U.S. Provisional Application No. 63/702,765, filed Oct. 3, 2024

### TECHNICAL FIELD

This invention relates to cell-free biocatalysis systems that convert inorganic pyrophosphate (PPi) into biochemical work by regenerating adenosine triphosphate (ATP), in situ, under controlled pH, dissolved magnesium ions (pMg), ionic strength, and temperature.

### BACKGROUND OF INVENTION

Using biological enzymes outside their natural cellular environments offers a significant and largely untapped opportunity to enhance bio-production processes. By decoupling enzymes from their native contexts, one can modify both their sequences and structures in ways that are favorable for industrial applications. This includes engineering enzymes to remove feedback inhibition caused by accumulated products, increasing their affinity for substrates, and improving their stability under conditions optimal for other enzymes in the system. Additionally, one can optimize and maintain reaction conditions that differ substantially from native cellular conditions to achieve higher efficiency. To date, however, such efforts have been substantially limited by the lack of scalable and cost-effective ways to energize biosynthetic reactions in cell-free conditions, and the present invention addresses this limitation.

ATP, phosphoenolpyruvate (PEP) and other energy intermediates are used in a variety of biochemical processes including high-energy phosphorylation of various molecules. However, another high-energy intermediate, PPi, has substantially lower cost, which is critical for cost-effectiveness of cell-free bioproduction. Additionally, PPi can be regenerated from phosphate using physical processes (such as calcination or microwave irradiation, etc.), thus creating a link between physical energy in form of heat and biochemical energy that enzymes can use for catalysis.

Phosphorylated molecules have very broad utility in industrial applications, medicine, nutrition, agriculture, and numerous domains of scientific research. In industry, they are used as flame retardants, detergents, and corrosion inhibitors. In medicine, they are pivotal in drug development as kinase inhibitors and antiviral agents. Nutritionally, they are essential for energy metabolism and are components of vital vitamins and coenzymes. In agriculture, they enhance crop yields as fertilizers and protect plants as pesticides. In scientific research, they are fundamental in molecular biology techniques, signal transduction studies, and biochemical assays.

### BRIEF DESCRIPTION OF INVENTION

Phosphorylation is essentially adding a phosphate group to a molecule, typically enzymatically. As in any enzymatic chemical reaction in cell-free phosphorylation the initial substrate molecules, enzymes, and energy molecules that are combined, for example, with the addition of pH buffers, salts, and water as auxiliary inputs. A chamber in which the reaction takes place (e.g. a reaction chamber) contains the constituents, and in conclusion, new products and some remaining reactants remain. The products are then formulated using, perhaps, filtering, freeze drying, and the like to end up with a stable form of the desired reaction product(s).

In its most basic embodiment, the method of the instant invention comprises mixing substrate molecules, PPi, and enzymes that utilize PPi (PPi-dependent enzymes) in a reaction chamber and ending with stable-form products, including phosphorylated molecules.

Other embodiments comprise capturing byproducts, processes for separating products, and PPi regeneration. By adding some of these sub-process steps, recaptured PPi can be reused for successive processes, and by adding other PPi-dependent enzymes, process speed and efficiency can be enhanced.

Provisional application, 63/702,765 describes a PPi-to-ATP energy transfer cycle that uses PPi-dependent phosphoenolpyruvate carboxykinase (PPi-PEPCK) to generate phosphoenolpyruvate (PEP) from oxaloacetate and PPi, and PPDK to convert PEP and AMP to ATP with adenylate kinase (AK) interconverting nucleotides, under reactor control of pH, pMg, ionic strength, and temperature, and with options for in-situ PPi regeneration and continuous operation.

To reduce substrate lability and cost and to coordinate redox needs, an optional aspartate dehydrogenase feeder can generate oxaloacetate in situ from L-aspartate while concomitantly forming nicotinamide adenine dinucleotide hydride (NADH) or nicotinamide adenine dinucleotide phosphate (NADPH) and releasing ammonium that counteracts acidification, improving robustness and economics while feeding the same PPi-PEPCK node disclosed in the provisional application, 63/702,765.

### BRIEF DESCRIPTION OF DRAWINGS

FIG. 1 illustrates a basic system based on using PPi and related enzymes.

FIG. 2 illustrates an embodiment wherein the process of FIG. 1 is embellished with additional enzymes and additional substrates.

FIG. 3 illustrates the system of FIG. 1 wherein byproducts are captured, products are separated, and phosphates are captured and used for PPi regeneration.

FIG. 4 shows another embodiment wherein PPi regeneration makes use of microwave energy and occurs in the reaction chamber or a connected chamber.

FIG. 5 shows an embodiment wherein PPi and PPi-PEPCK are inputs to the process along with oxaloacetate resulting in carbon dioxide (CO₂) as a byproduct, along with phosphate and PEP as separated end products.

FIG. 6 shows an embodiment wherein PPi-RK enzyme along with ribose are used as inputs and ribose-5-phosphate is one of the separated end products.

FIG. 7 shows an embodiment with an extensive set of input constituents resulting in ATP and pyruvate as separated end products.

FIG. 8 shows an embodiment with an extensive set of input constituents resulting in ATP among the separated end products.

FIG. 9 is a table showing thermodynamic properties of individual reactions and the net reaction of energy transfer from PPi to ATP under specific conditions.

FIG. 10 is a schematic depiction of the process where PPi-to-ATP energy transfer pathway operates in the reaction chamber to provide a replenished source of ATP by regenerating ADP and AMP into ATP using the energy potential of the pyrophosphate.

FIG. 11 represents the PPi-to-ATP energy cycle.

FIG. 12 shows a modification to the cycle shown in FIG. 11 wherein additional efficiency can be gained in the PPi-to-ATP energy cycle.

FIG. 13 shows a specific instance of the FIG. 11 wherein PPi-to-ATP energy cycle supplies the energy for the ligase enzymes that produce of an antimicrobial peptide Phonsphonalamide A in cell-free conditions.

### DETAILED DESCRIPTION OF INVENTION

ATP, PEP and other energy intermediates are used in a variety of biochemical processes including high-energy phosphorylation of various molecules. However, PPi has advantages for lower cost, and higher reaction efficiency compared to traditional energy molecules.

Here is a breakdown of the cyclic pathway for four core enzymes:

For pyrophosphate-dependent phosphoenolpyruvate carboxykinase (PEPCK), the reaction is: Oxaloacetate+PPi→Phosphoenolpyruvate+CO2+Pi wherein oxaloacetate is converted to PEP using PPi as an energy source. For pyruvate phosphate dikinase (PPDK), the reaction is Phosphoenolpyruvate+AMP+PPi→Pyruvate+ATP+Pi wherein PEP is converted to pyruvate while regenerating ATP directly from AMP. Most other routes do not have this advantage and go the less energetically efficient two-step route of AMP=>ADP, then ADP=>ATP. With PPDK one achieves it with one step by leveraging the excess of energy in PEP plus the energy of PPi to make ATP directly from AMP. Many biosynthetic reactions, such as amino-acyl-tRNA ligases, non-ribosomal peptide synthases, polyketide synthases, produce AMP and PPi as product rather than ADP and Pi and our cycle is uniquely well positioned to recycle AMP and PPi efficiently.

For pyruvate carboxylases, the reaction is Pyruvate+CO2+ATP→Oxaloacetate+ADP+Pi wherein pyruvate is carboxylated to form oxaloacetate using ATP and storing excess of energy in form of ADP.

For adenylate kinase, the reaction is 2 ADP→ATP+AMP where adenosine phosphates are interconverted to maintain energy balance.

Optionally, carbonic anhydrase from the reaction CO2+H2O→HCO3(−)+H(+) facilitates CO₂ solubility in water, while an ATP hydrolysis reaction, ATP+H2O→ADP+Pi, illustrates an ATP-consuming process.

The foregoing is believed to be a novel cyclic pathway for energy transfer from PPi to nucleoside phosphates in a cell-free setting and underpins this invention process.

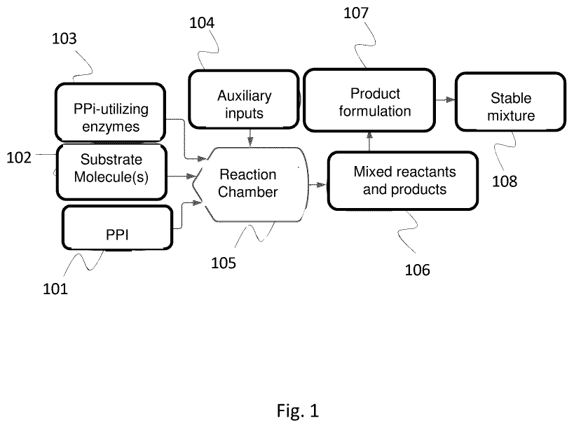

As shown in FIG. 1, in one embodiment, the inputs comprise PPi (101) as an energy source (i.e. energy-rich molecule with high-energy phosphate-phosphate bond), which binds a PPi-dependent enzyme (103) and releases its energy to produce phosphorylated form of the input substrate (102). The process occurs in a reaction chamber (105) where the inputs are combined. The reaction chamber may include reaction control devices such as mixing mechanism(s), sensor(s), and titration equipment that may offer more precise control and maintenance of desired reaction conditions. The inputs may include but are not limited to the active form of the PPi-dependent enzyme, substrate molecule(s) including PPi and auxiliary inputs—FIG. 1, 104—such as pH buffering agent(s), salt(s) of required metal(s), water or another solvent, and the like. Certain byproduct(s) of the enzymatic reaction may spontaneously leave the reaction medium due to low solubility in the medium.

An important aspect of the present invention is the control of the pMg, temperature, pH, and ionic strength, which are critical factors influencing the efficiency of the process.

The desired pMg concentration may be controlled, for example, via auxiliary inputs or reaction control devices such as titration equipment. These control mechanisms allow for precise adjustment of magnesium ion concentrations, ensuring that the pMg remains within the optimal range throughout the process.

The optimal values of pMg range from about 1 to about 5, wherein pMg denotes the negative logarithm (base 10) of the free magnesium ion activity in the system i.e., pMg=−log₁₀(a_Mg²⁺)

The optimal values of pH range from about 1 to about 14, wherein pH denotes the negative logarithm (base 10) of the hydrogen ion concentration in the system.

The optimal values of temperature range from about 5 degrees Celsius to about 95 degrees Celsius.

The optimal values of ionic strength of the solution range from about 0.01 to 1M.

When product formation slows down or halts the reaction medium (consisting of the reactants, products, enzyme(s), FIG. 1, 106, and auxiliary inputs) may be subject to downstream processing steps. Those steps may separate phosphate (e.g. for regeneration back to PPi), the phosphorylated product(s), and other molecule(s) formed as product(s) or byproduct(s) in the reaction chamber. The product formulation may separate macromolecules (e.g. the enzyme(s)), insoluble residues, and transform the reaction mixture into dry form for greater stability (107 and 108). Phosphate, if separated, may be converted back to pyrophosphate and reused in the subsequent reactions as energy input.

In some cases, the process may be run continuously with inflow of the inputs to the reaction chamber and outflow of the reaction mixture for downstream processing, and optionally, partial or full recycling of the byproducts into the inputs (e.g. as phosphate calcination to produce pyrophosphate) to be used as the energy input.

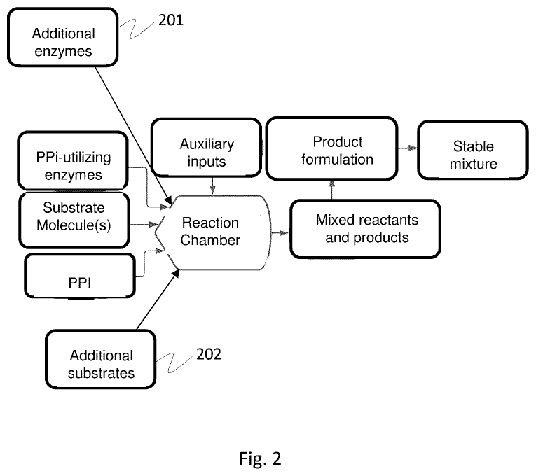

In FIG. 2, the process on FIG. 1 is augmented with additional substrate(s) (202) and enzymes(s) (201), added to form additional product(s). In some embodiments such product(s) may be phosphorylated. In other embodiments the product(s) may be non-phosphorylated but require phosphorylated intermediates, or energy derived from phosphorylated molecules, for their production.

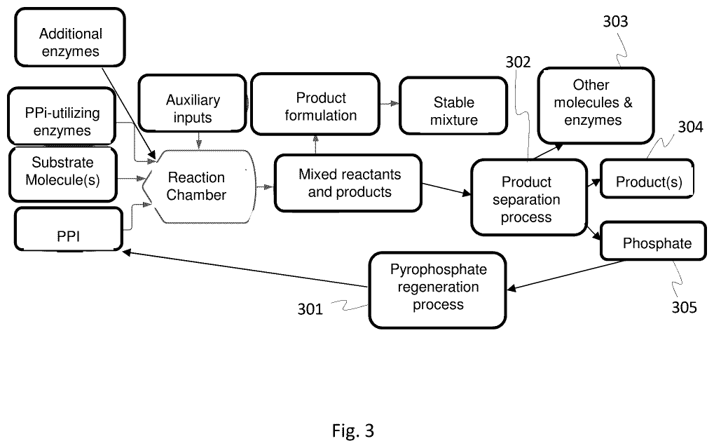

FIG. 3 is the process of FIG. 1 now set for byproduct(s) capture (301), product separation (302), and the end result of other molecules and enzyme(s) (303), product(s) (304) and phosphate (305) which is then used in regeneration to provide PPi input for subsequent operation.

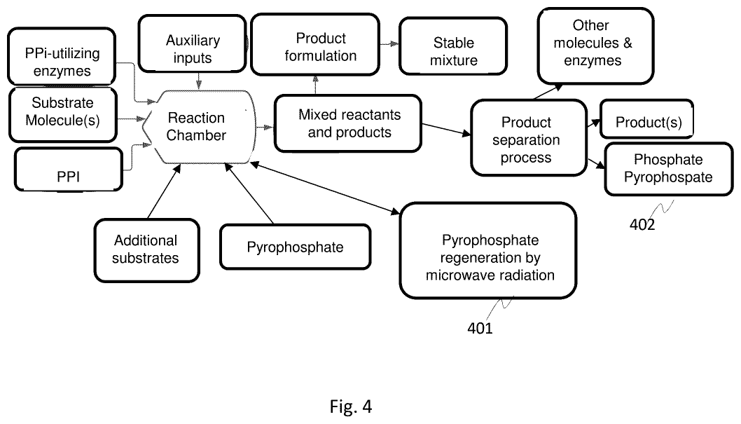

In FIG. 4 regeneration of the pyrophosphate from phosphate (402) occurs in the reaction chamber or a connected chamber and is energized through microwave radiation (401). This method offers the ability to lower (energy-depleted) phosphate concentration while raising (energy-containing) pyrophosphate concentration without halting the reaction thus conferring additional thermodynamic driving force to the enzymatic reaction(s). Alternatively, such a pyrophosphate regeneration method may be a faster, less hazardous, and less energy-intensive alternative than calcination, for example. Such method may also provide continuous versions of the process with (partial or complete) re-use of phosphate as energy carrier.

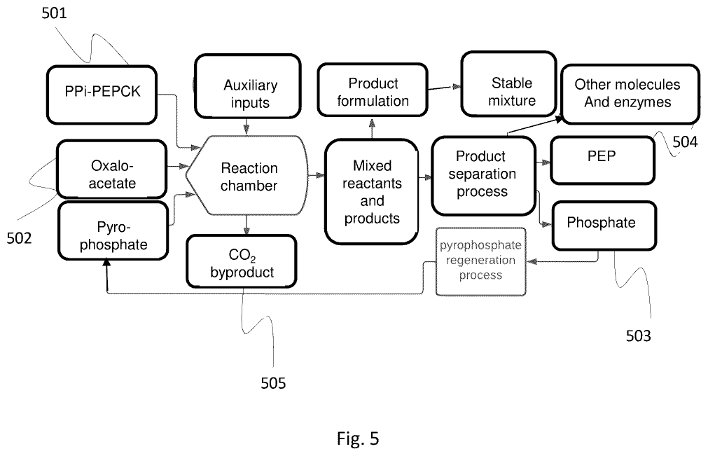

In the embodiment shown in FIG. 5, the PPi-dependent enzyme is phosphoenolpyruvate carboxykinase (PPi-PEPCK), 501, the inputs are oxaloacetate, 502, and PPi, while the byproduct with limited solubility in the aqueous reaction medium is CO2, 505. The desired phosphorylated product is PEP (504) and the byproduct is phosphate (503). The enzymatic reaction catalyzed by the PPi-PEPCK enzyme is: oxaloacetate+PPi⇔PEP+phosphate+CO2. In some embodiments PEP and/or phosphate may be separated from the reaction mixture. In some other embodiments the reaction mixture may be filtered to separate macromolecules (e.g. the enzyme) and then transformed into a dry mixture of reactants and products.

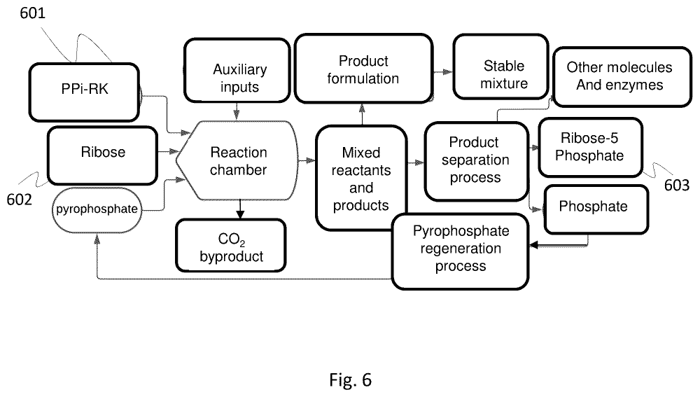

In the embodiment of FIG. 6, the PPi-dependent enzyme is ribokinase (PPi-RK), 601, the inputs are ribose, 602, and PPi, while the desired phosphorylated product is ribose-5-phosphate (603) and/or ribose-1-phosphate (R5P and/or R1P) and the byproduct is phosphate. The enzymatic reaction catalyzed by the PPi-RK enzyme is: ribose+PPi⇔ribose-phosphate+phosphate. In some embodiments ribose-phosphate(s) and/or phosphate may be separated from the reaction mixture. In some other embodiments the reaction mixture may be filtered to separate macromolecules (e.g. the enzyme) and then transformed into a dry mixture of reactants and products. In some other embodiments additional enzymes can be added to transform ribose-phosphates into other desired molecules. For instance, if ribose, adenine and oxaloacetate used as inputs along with the PPDK, PPi-PEPCK, ribose-phosphate diphosphokinase (RPDK), and adenine phosphoribosyltransferase (APRT) enzymes, ribose-5-phosphate would be further converted into ATP. The enzymatic reaction catalyzed by the PPDK enzyme is: AMP+PPi+PEP⇔ATP+phosphate+pyruvate. The enzymatic reaction catalyzed by the RPDK enzyme is: ATP+ribose-5-phosphate=AMP+PRPP. The enzymatic reaction catalyzed by the APRT enzyme is: adenine+PRPP⇔AMP+PPi. In some other embodiments adenosine as the input may be substituted with other nucleoside or deoxynucleoside to produce respective (deoxy)nucleoside monophosphate using the enzymes that naturally phosphorylate such substrate or are engineered to have such activity. Additionally, some embodiments may produce (deoxy)nucleoside diphosphate, triphosphate or a mixture of (deoxy)nucleoside mono-, di-, and triphosphates by addition of the PPi-PEPCK, pyruvate phosphate dikinase (PPDK), and adenylate kinase (AK) enzymes as described hereafter. In some other embodiments a mixture of nucleoside(s) and/or deoxynucleoside(s) may be used as input(s) to produce a mixture of their corresponding (deoxy)nucleoside mono-, di-, and triphosphates.

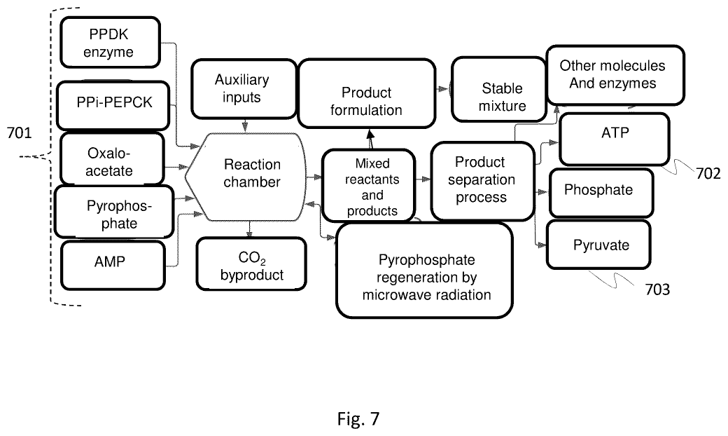

In the embodiment of FIG. 7, the PPi-dependent enzyme is phosphoenolpyruvate carboxykinase (PPi-PEPCK), and additional enzyme is pyruvate phosphate dikinase (PPDK). The inputs are adenosine-5-monophosphate (AMP), oxaloacetate, and PPi (701), while the byproduct with limited solubility in water is CO2. The desired phosphorylated product is adenosine-5-triphosphate (ATP), 702, and the byproducts are phosphate and pyruvate (703). The enzymatic reaction catalyzed by the PPi-PEPCK enzyme is: oxaloacetate+PPi⇔PEP+phosphate+CO2. The enzymatic reaction catalyzed by the PPDK enzyme is: AMP+PPi+PEP⇔ATP+phosphate+pyruvate. In some embodiments the AMP input may be produced as described above from adenine and ribose. In some other embodiments, input AMP or other nucleoside-5-monophosphates may be derived from enzymatic cleavage of RNA by RNA-exonuclease(s) and input dAMP or other (deoxy-)nucleoside-5-monophosphates may be derived from enzymatic cleavage of DNA by DNA-exonucleases. In some embodiments suitable endonucleases, helicases, or other auxiliary enzymes may be combined with exonucleases for increased output of the (deoxy)nucleoside-5-phosphates.

In some embodiments ATP and/or phosphate and/or pyruvate may be separated from the reaction mixture. In some other embodiments the reaction mixture may be filtered, for instance using tangential flow filtration (TFF), to separate macromolecules (e.g. the enzymes) and then transformed into a dry mixture of reactants and products.

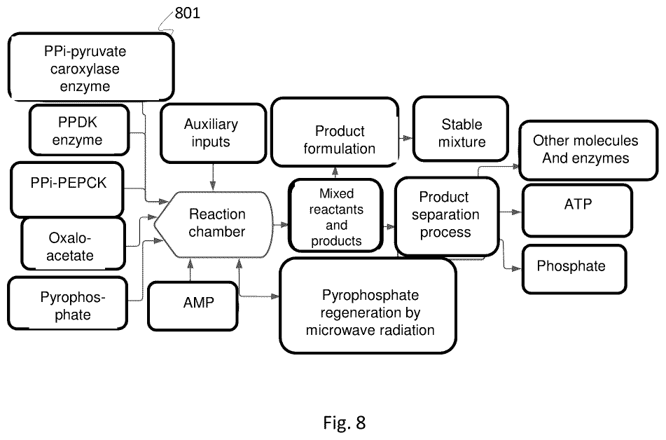

FIG. 8 is a version of the process on FIG. 7 where an active form of the PPi-dependent pyruvate carboxylase (801) is added in order to recapture CO2 byproduct of the PEPCK enzyme and regenerate oxaloacetate using engineered form of the pyruvate carboxylase enzyme, which can utilize PPi instead of ATP in the following reaction: PPi+pyruvate+CO2⇔oxaloacetate+phosphate. ATP-dependent enzymes for pyruvate carboxylase exist naturally ⇔ and need to be engineered towards using PPi as an energy source instead of ATP. Alternatively, natural or engineered forms of ATP-dependent pyruvate carboxylase enzymes can be used to regenerate oxaloacetate from pyruvate, ATP (products of the PPDK), and CO₂ (product of PPi-PEPCK) in the following reaction: ATP+pyruvate+CO2⇔ADP+oxaloacetate+phosphate. In such case, residual energy present in ADP can be accessed by adenylate kinase (AK) enzyme that interconverts nucleoside phosphates in the following reaction: 2 ADP⇔ATP+AMP. This reaction serves both as a source of AMP for the PPDK enzyme and the balancing mechanism to ensure the production of nucleoside phosphates is balanced with the demand for them created by the ATP- or ADP-dependent enzymes.

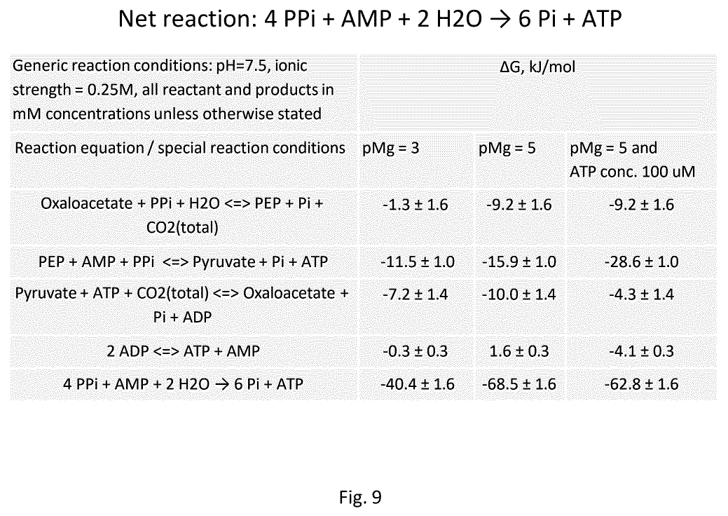

FIG. 9 illustrates existence of the conditions in which the four core reactions are thermodynamically feasible simultaneously (the right-most column). The last row shows the net reactions that summarizes the four core reactions (cancelling out the intermediates that are produced and consumed in equal amounts within the cycle), and shows overall thermodynamic feasibility of the cycle as a vehicle for energy transfer from pyrophosphate to nucleoside phosphates (e.g. ATP/ADP/AMP or dATP/dADP/dAMP, or GTP/GDP/GMP, etc.) Optionally, carbonic anhydrase enzyme or supplementation of bicarbonate may be used to increase flux through the pyruvate carboxylase reaction.

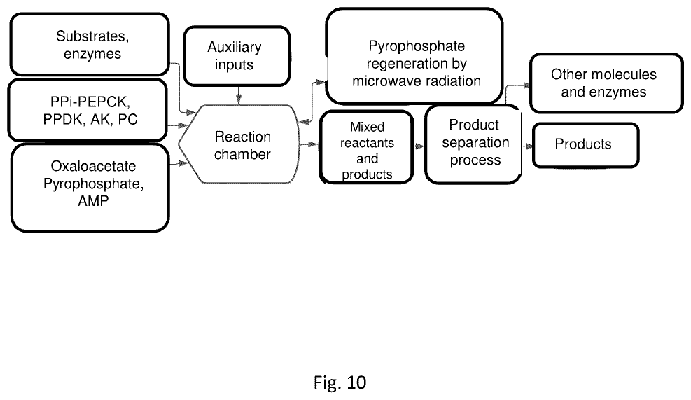

FIG. 10 represents the process where a PPi-to-ATP energy transfer pathway provides a replenished source of ATP by regenerating ADP and AMP into ATP using the pyrophosphate's energy potential. That energy is used, in part, to transform substrates into products using enzymes that use ATP or ADP as the phosphate or energy donor.

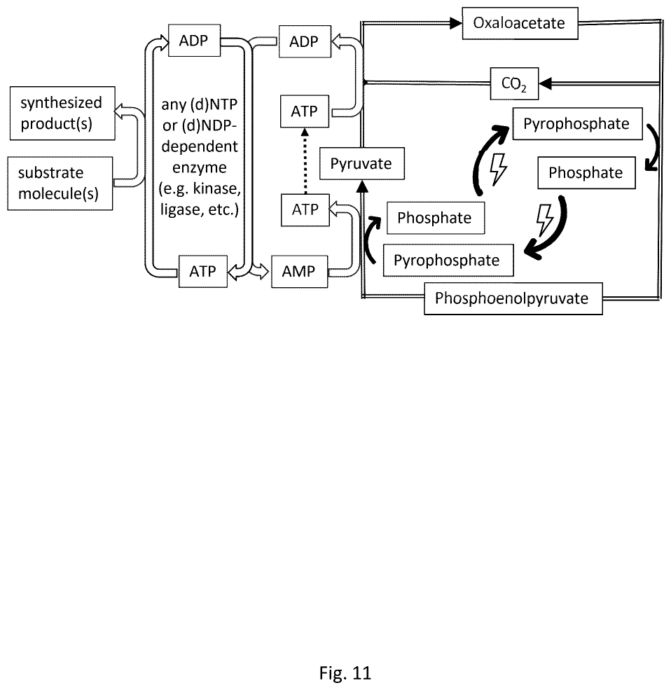

FIG. 11 represents the PPi-to-ATP energy cycle. The double line traces the carbon cycle (oxaloacetate=>PEP=>pyruvate=>oxaloacetate). The thick open arrows trace the energy cycle (AMP<=>ATP<=>ADP) that may be coupled to the other enzymes that use such cycle as replenishable source of phosphorylation and/or energy substrates. The thick black arrows trace the cycle of phosphates (pyrophosphate<=>2 phosphate). In some embodiments phosphate cycle may remain open and require supplementation of pyrophosphate (e.g. if the product(s) are phosphorylated and their removal depletes system of phosphates). In other embodiments the phosphate cycle can be closed with all free phosphate recycled back into pyrophosphate. In some embodiments such recycling may occur directly within the reaction chamber or in an adjacent chamber (e.g. by means of microwave radiation) to energize the cycle while it is in operation. Other means to achieve conversion of phosphate into pyrophosphate exist and may be used. Those include but are not limited to: 1) High-Energy Processes such as Thermal Dehydration (Calcination), Photochemical Methods (UV Irradiation), High-Energy Radiation (Gamma Irradiation), Microwave Irradiation, Plasma Methods, Electrochemical Methods, Hydrothermal Synthesis, Ultrasonic Cavitation, etc.; 2) Chemical Dehydration Using Dehydrating Agents such as Phosphorus Oxychloride (POCl₃) and Thionyl Chloride (SOCl₂), Carbodiimide-Mediated Condensation, Carbonyldiimidazole (CDI) Activation, Condensing Agents (e.g. Cyanamide, Imidazole), Phosphorylating Reagents (Phosphoryl Azide), Chemical Synthesis via Anhydride Formation or Formation of Phosphoanhydride Bonds, etc; 3) Catalytic processes using physical or biological catalysts such as Polyphosphate Kinase (PPK), Mineral Surfaces, Metal Ion Catalysis, and so on.

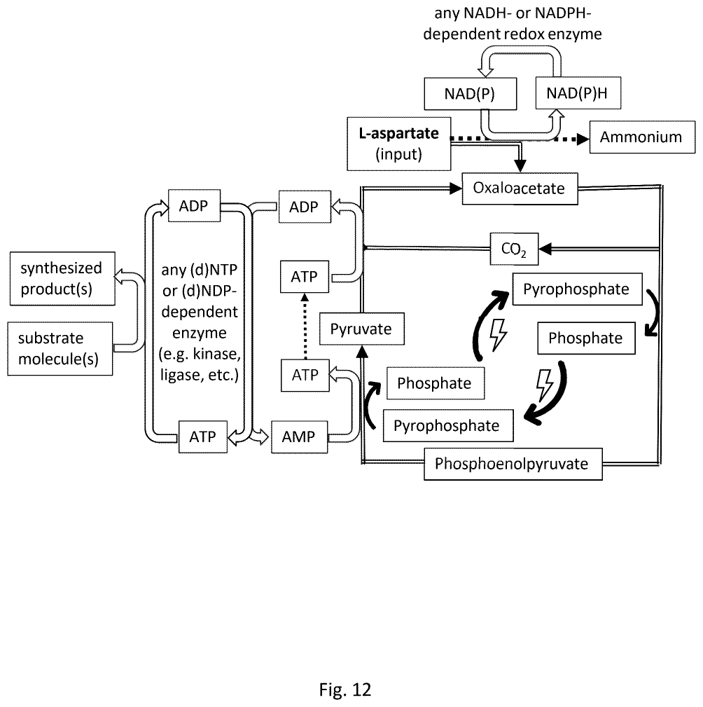

In FIG. 12, the cycle shown in FIG. 11 has been modified wherein an optional aspartate dehydrogenase feeder can generate oxaloacetate in situ from L-aspartate while concomitantly forming nicotinamide adenine dinucleotide hydride (NADH), or nicotinamide adenine dinucleotide phosphate (NADPH), and releasing ammonium that counteracts acidification, improving robustness and economics while feeding the same PPi-PEPCK node disclosed in the provisional.

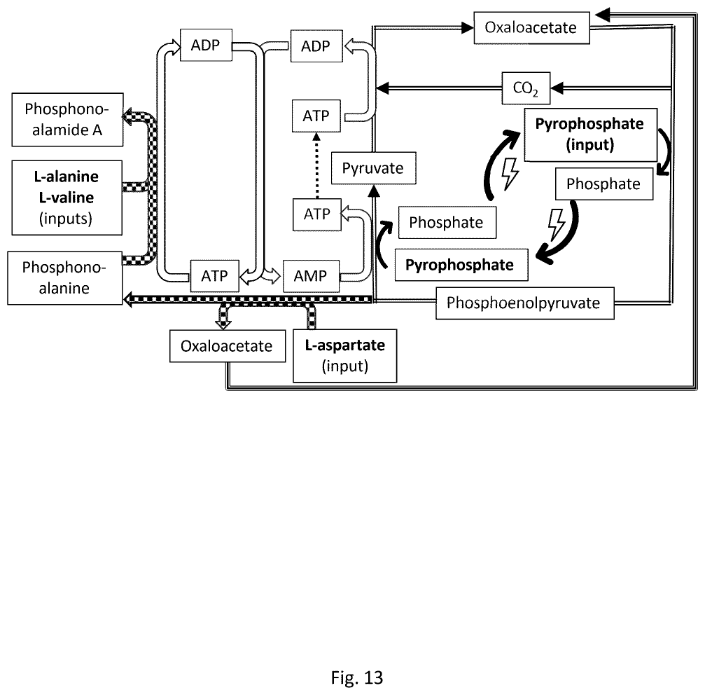

In FIG. 13 the PPi-to-ATP energy cycle starts with L-aspartate, which is converted to oxaloacetate by phosphonopyruvate aminotransferase enzyme and in the same reaction PEP is used as an acceptor of the amine group forming L-phosphonoalanine, non-canonical amino acid and key component of all phosphoalamide peptides. As an example, figure shows synthesis of the phosphonoalamide A from L-phosphonoalanine externally supplied L-alanine and L-valine. By supplying other, canonical or non-canonical amino acids many other L-phosphonoalanine-containing peptides can be synthesized with the natural or engineered ligase enzymes.

In the interest of further clarity, the following descriptions are offered. The reaction chamber includes mixing, sensors, and titration to regulate pH, pMg, ionic strength, and temperature within ranges disclosed in the provisional (pH 1-14, pMg 1-5, ionic strength 0.01-1 M, temperature 5-95° C.) with active magnesium titration to maintain free Mg(2+) activity for enol phosphate and nucleotide complexes and to stabilize equilibria during fed-batch or continuous operation as described. Exemplary compositions for combined PPi-PEPCK+PEP-to-ATP operation include 50-150 mM HEPES or ammonium bicarbonate buffer, 1-10 mM NaCl, 1-5 mM DTT, 0.5-2 mM MgCl2, 0.05-0.2 mM FeCl3, 1-10 mM PPi, and 1-10 mM oxaloacetate or L-aspartate, run at room temperature under mixing for at least two hours, which are within the optimal conditions tested.

PPi-PEPCK converts oxaloacetate and PPi to PEP and phosphate with release of CO2, and the step is favored by appropriate pH/pMg and ionic strength with CO2 handling optionally aided by carbonic anhydrase or bicarbonate supplementation as disclosed in the provisional text and figures, and is operable under the representative composition above to provide PEP continuously to the downstream module.

The PEP-to-ATP module is selectable between PPDK acting on PEP+AMP+PPi and PK acting on PEP+ADP, each integrated with adenylate kinase interconverting 2 ADP⇔ATP+AMP to sustain nucleotide balance as ATP is consumed by downstream ATP-dependent enzymes, with selection based on substrate availability and process goals as described here to generalize the provisional's PPDK-centric pathway. The combined PPi-PEPCK+PPDK (and AK) operation under the representative composition above yielded PEP, ATP, ADP, AMP, pyruvate, and phosphate detectable by LC/MS and luciferase assays as recorded in the supplied enablement data, and a PK-based variant is configured analogously with ADP as the direct PEP acceptor while AK manages ATP/ADP/AMP pools during ATP consumption by product-forming enzymes.

In closed-loop mode, pyruvate, ATP, and CO2 are converted to oxaloacetate and ADP via a pyruvate carboxylase (ATP-dependent) or an engineered PPi-dependent pyruvate carboxylase variant, thereby regenerating oxaloacetate to feed PPi-PEPCK and reducing pyruvate accumulation as shown in FIG. 8 and the surrounding text, while open-loop mode omits the carboxylation and tolerates pyruvate accumulation and removal or secondary conversions as disclosed in the provisional. Both modes are compatible with the selectable PEP-to-ATP module and the representative compositions and controls described above, including continuous operation with inflow of PPi, substrates, and enzymes and outflow to separations and recycle.

An optional feeder generates oxaloacetate from L-aspartate using an aspartate dehydrogenase selected from NAD-dependent and NADP-dependent classes to simultaneously produce NADH or NADPH and release ammonium that counteracts acidification, enabling the use of more stable and lower-cost L-aspartate while co-supplying reduced cofactors to other modules and feeding oxaloacetate directly into PPi-PEPCK in the same controlled environment, thereby improving robustness and economics without altering the cycle logic, and this module is inserted upstream of the PPi-PEPCK node in FIGS. 5-11 without requiring new schematics.

PPi is optionally regenerated from phosphate within the reaction chamber or a connected chamber using microwave irradiation as disclosed, with alternative physical and chemical pathways enumerated in the provisional, allowing continuous restoration of PPi and lowering phosphate to improve driving force while the biocatalysis continues under the same control regime and separation schemes, which is compatible with the optional aspartate dehydrogenase feeder and either PEP-to-ATP module as configured here.

Representative combined conditions for PPi-PEPCK+PPDK operation were room temperature for at least 2 hours with 100 mM HEPES pH 7.5 or 100 mM ammonium bicarbonate, 10 mM NaCl, 5 mM DTT, 5 mM PPi, 1 mM MgCl2, 0.1 mM FeCl3, 5 mM oxaloacetate, and 5 mM nucleotide mix, yielding PEP, ATP, ADP, and AMP as detected by LC/MS and luciferase assays under the listed compositions, with analogous operation feasible for PK-based PEP-to-ATP by substituting ADP for AMP at the ATP-generating step while maintaining AK balancing in the same mixture. The supplied data also include ribose-5-phosphate and pseudo-UMP formation using ATP-dependent RK and pseudo-UMP-glycosidase with ribose and uracil under similar conditions, as well as CTP regeneration from CMP using PEP and promiscuous PPDK activity, each listing inputs, enzymes, and detected products, which are integrated here as use-case confirmations of operability and scope.

Microwave-driven dehydration of dried NaH2PO4 in a quartz crucible using 1-minute pulses increased PPi production up to at least the seventh pulse with potential for further conversion as described, and luciferase-based assays run under the same representative conditions as the PPi-PEPCK+PPDK combo confirmed PPi presence, supporting the continuous PPi regeneration option disclosed here.

The regenerated ATP supplies ATP-dependent enzymes such as ligases, synthetases, kinases, polymerases, carboxylases, and transferases, with ATP-grasp ligases used to exemplify peptide ligations (dipeptide formation and coupling to L-phosphonoalanine) yielding phosphonoalamides including phosphonoalamide A (FIG. 13). This is a use-case embodiment powered by the PPi platform rather than claimed as composition-of-matter.

Some objectives underlying this synthesis system comprise energy neutrality or exothermic energy release; and using cell-free processes that allow pH, temperature and ionic strength for optimal enzyme activity and stability.

Disclosed herein is a method for engineering nucleoside-phosphate-dependent enzymes to become pyrophosphate (PPi)-dependent by modifying their nucleoside-phosphate-binding pockets. This modification involves altering specific amino acid residues within the binding site to create steric hindrance that prevents the accommodation of nucleoside-phosphate molecules while allowing the binding of smaller pyrophosphate molecules. Such alterations can include introducing amino acids with bulkier side chains or adjusting the electrostatic environment within the pocket to selectively exclude nucleoside-phosphates. These engineering efforts can be further informed by comparing nucleoside-phosphate-dependent and PPi-dependent variants of natural enzymes whenever such are known. By analyzing commonalities and dissimilarities in the sequences and structures of these enzyme pairs, insights can be gained into critical residues and conformational features that influence substrate specificity. By redesigning the binding pocket in this manner, the enzyme's dependency shifts from nucleoside to pyrophosphate, thereby changing its substrate specificity and further utility for the systems described herein.

By way of example and without limitation, PPi-dependence is engineered by preserving the conserved Lys/Arg and Mg2+ network that coordinates the of beta-and gamma-phosphate group (shared by NTPs and PPi), while remodeling amino acids that recognize the nucleoside and alpha-phosphate, thereby maintaining the catalytic phosphoryl-transfer scaffold and shifting selectivity toward PPi as phosphate donor. Engineering steps include: (i) identifying residues mediating adenine/ribose recognition (e.g., aromatic stacking and hydrogen-bonding side chains) and alpha-phosphate clamps, and (ii) introducing bulkier and/or hydrophobic or neutral substitutions to occlude the nucleoside pocket and weaken α-phosphate binding, while (iii) retaining or conservatively re-orienting (e.g., Lys↔Arg) the of beta and gamma Lys/Arg network and metal-coordination geometry to optimize PPi chelation without loss of transition-state stabilization. The design can be optionally confirmed by structural modeling or docking of PPi and ATP in the modified active site to verify maintenance of beta- and gamma-coordination and exclusion of the nucleoside moiety. Activity is then assayed in a cell-free buffer with controlled pH, pMg, ionic strength, and temperature (e.g., HEPES or ammonium bicarbonate with automated Mg2+ titration to maintain pMg), using steady-state kinetics to determine k_{cat}/Kₘ, endpoint LC-MS conversions, and phosphate/PPi-coupled luminescence assays. A shift of at least about 5- to 10-fold in catalytic efficiency toward PPi (relative to ATP or other NTPs) is indicative of PPi-dependence for the engineered variant, with equivalent strategies that achieve the same functional outcome, including without limitation AI-guided protein-ligand design solutions such as LigandMPNN, PoketGet, or similar, being contemplated.

What is claimed is:

1. A system comprising:
a reaction chamber;
a plurality of PPi-utilizing enzymes;
substrate molecules;
PPi;
cofactors; and
metal ions,

2. The system as in claim 1 further comprising:
active forms of enzymes other than PPi-utilizing enzymes; and
additional substrates.

3. The system as in claim 1 further comprising:
a product-separation process.

4. The system in claim 2 further comprising:
pyrophosphate regeneration from phosphate using microwave radiation.

5. The system in claim 1 wherein:
active form of PPi-PEPCK enzymes replace active form of PPi-utilizing enzymes; and
oxaloacetate replaces substrate molecules.

6. The system as in claim 1 wherein:
PPi-utilizing enzyme is engineered to use PPi instead of natural phosphate donor.

7. The system as in claim 5 further comprising:
active form of PPDK enzymes; and
any one of of the group of guanosine monophosphate (GMP), cytidine monophosphate (CMP), uridine monophosphate (UMP), thymidine monophosphate (TMP), integral membrane protein (IMP), and pseudo-UMP and derivatives.

8. The system as in claim 7 further comprising:
active form of PPi-pyruvate carboxylase enzyme; and
active carbonic anhydrase operative to increase flux through pyruvate carboxylase and stabilize pH.

9. A cell-free method comprising:
converting oxaloacetate and PPi to phosphoenolpyruvate and phosphate with release of CO2.

10. The cell-free method of claim 9 wherein:
utilizing phosphoenolpyruvate in the PEP-to-ATP module.

11. The method of claim 9 wherein:
carboxylation is catalyzed by an ATP-dependent pyruvate carboxylase to close the oxaloacetate-pyruvate loop, thereby regenerating oxaloacetate for PPi-PEPCK and reducing pyruvate accumulation.

12. The system of claim 1 wherein:
the PPi-driven ATP module comprises PPi-dependent phosphoenolpyruvate carboxykinase, adenylate kinase, and a PEP-to-ATP unit selected from pyruvate kinase and pyruvate phosphate dikinase, and further comprises sensors and titration means to regulate pH and pMg during operation.
